(19) United States
(12) Patent Application Publication
(10) Pub. No.: US 2024/0349993 A1
(43) Pub. Date: Oct. 24, 2024

(54) SELECTIVE USE OF DIFFERENT VIDEO STREAMS GENERATED BY AN IMAGING DEVICE TO PERFORM AN IMAGE-BASED OPERATION

(71) Applicant: Intuitive Surgical Operations, Inc., Sunnyvale, CA (US)

(72) Inventors: Maximilian H. Allan, Mill Valley, CA (US); Mahdi Azizian, San Jose, CA (US); Thomas Kurmann, Bern (CH)

(21) Appl. No.: 18/685,892

(22) PCT Filed: Sep. 2, 2022
(86) PCT No.: PCT/US2022/042528

§ 371 Date: Feb. 22, 2024

Related U.S. Application Data
(60) Provisional application No. 63/241,443, filed on Sep. 7, 2021.

(51) Int. Cl. A61B 1/00; A61B 1/12

(52) U.S. Cl. CPC A61B 1/00193; A61B 1/000094; A61B 1/000096; A61B 1/126

(57) ABSTRACT

An illustrative image processing system is configured to apply. as an input to a processing module. a first video stream generated by an imaging device during a medical procedure. The processing module is configured to generate. based on the input. output data used to perform an image-based operation associated with the medical procedure. The image processing system is further configured to detect, while the first video stream is being applied to the processing module. a deficiency associated with the first video stream and apply. as the input to the processing module and based on the detecting of the deficiency. a second video stream generated by the imaging device during the medical procedure.

### RELATED APPLICATIONS

The present application claims priority to U.S. Provisional Patent Application No. 63/241,443, filed Sep. 7, 2021, the contents of which is hereby incorporated by reference in its entirety.

### BACKGROUND INFORMATION

A stereoscopic imaging device (e.g., a stereoscopic endoscope) used during a medical procedure may output two video streams, e.g., a first video stream intended to be presented to a left eye of a user and a second video stream intended to be presented to a right eye of the user. In this manner, the viewer may view a scene captured by the imaging device in three dimensions (3D).

In some instances, it may be desirable for an image processing system to use one or both of the video streams output by a stereoscopic imaging device to perform one or more image processing operations. For example, one or both of the video streams may be analyzed by the image processing system to recognize an object depicted in the video streams, classify a particular image frame included in the video streams as being of a certain type, etc. Unfortunately, the performance of such image processing operations may be degraded by the presence of debris, such as tissue, blood, or water, which might be on one or both lenses of the stereoscopic imaging device.

### SUMMARY

The following description presents a simplified summary of one or more aspects of the systems and methods described herein. This summary is not an extensive overview of all contemplated aspects and is intended to neither identify key or critical elements of all aspects nor delineate the scope of any or all aspects. Its sole purpose is to present one or more aspects of the systems and methods described herein as a prelude to the detailed description that is presented below.

An illustrative system includes a memory storing instructions and a processor communicatively coupled to the memory and configured to execute the instructions to: apply, as an input to a processing module, a first video stream generated by an imaging device during a medical procedure, the processing module configured to generate, based on the input, output data used to perform an image-based operation associated with the medical procedure; detect, while the first video stream is being applied to the processing module, a deficiency associated with the first video stream; and apply, as the input to the processing module and based on the detecting of the deficiency, a second video stream generated by the imaging device during the medical procedure.

An illustrative system includes a memory storing instructions and a processor communicatively coupled to the memory and configured to execute the instructions to: monitor first and second video streams generated by an imaging device during a medical procedure for one or more deficiencies, the first video stream generated by the imaging device using a first image capture assembly and the second video stream generated by the imaging device using a second image capture assembly; and selectively use, based on the monitoring, one or more of the first video stream or the second video stream to perform an image-based operation associated with the medical procedure.

An illustrative method includes applying, by an image processing system as an input to a processing module, a first video stream generated by an imaging device during a medical procedure, the processing module configured to generate, based on the input, output data used to perform an image-based operation associated with the medical procedure; detecting, by the image processing system while the first video stream is being applied to the processing module, a deficiency associated with the first video stream; and applying, by the image processing system as the input to the processing module and based on the detecting of the deficiency, a second video stream generated by the imaging device during the medical procedure.

An illustrative non-transitory computer-readable medium may store instructions that, when executed, direct a processor of a computing device to: apply, as an input to a processing module, a first video stream generated by an imaging device during a medical procedure, the processing module configured to generate, based on the input, output data used to perform an image-based operation associated with the medical procedure; detect, while the first video stream is being applied to the processing module, a deficiency associated with the first video stream; and apply, as the input to the processing module and based on the detecting of the deficiency, a second video stream generated by the imaging device during the medical procedure.

### BRIEF DESCRIPTION OF THE DRAWINGS

The accompanying drawings illustrate various embodiments and are a part of the specification. The illustrated embodiments are merely examples and do not limit the scope of the disclosure. Throughout the drawings, identical or similar reference numbers designate identical or similar elements.

FIG. 1 shows an illustrative medical imaging system.

FIG. 2 shows an illustrative implementation of the image processing system of FIG. 1.

FIGS. 3-8 show illustrative video stream selection heuristics.

FIG. 9 shows an illustrative computer-assisted medical system according to principles described herein.

FIG. 10 shows an illustrative computing system according to principles described herein.

### DETAILED DESCRIPTION

An illustrative image processing system is configured to monitor first and

second video streams generated by an imaging device during a medical procedure for one or more deficiencies and selectively use, based on the monitoring, the first video stream and/or the second video stream to perform an image-based operation associated with the medical procedure.

For example, the image processing system may be configured to apply, as an input to a processing module, a first video stream generated by an imaging device during a medical procedure. As described herein, the processing module may be configured to generate, based on the input, output data used to perform an image-based operation associated with the medical procedure. The image processing system may be further configured to detect, while the first video stream is being applied to the processing module, a deficiency associated with the first video stream and apply, as the input to the processing module and based on the detecting of the deficiency, a second video stream generated by the imaging device during the medical procedure. As described herein, the second video stream may be applied as the input in place of or in addition to the first video stream.

As another example, the processing module may implement a stereoscopic (or “stereo”) algorithm that requires the use of both video streams output by the imaging device. If a deficiency is detected in one of the video streams, the stereo algorithm may be stopped (e.g., until the deficiency is resolved) or modified (e.g., a monoscopic (or “mono”) view may be used in place of a stereo view until the deficiency is resolved).

The principles described herein may result in improved image-based operations being performed compared to conventional techniques that do not selectively use one or both video streams output by an imaging device, as well as provide other benefits as described herein.

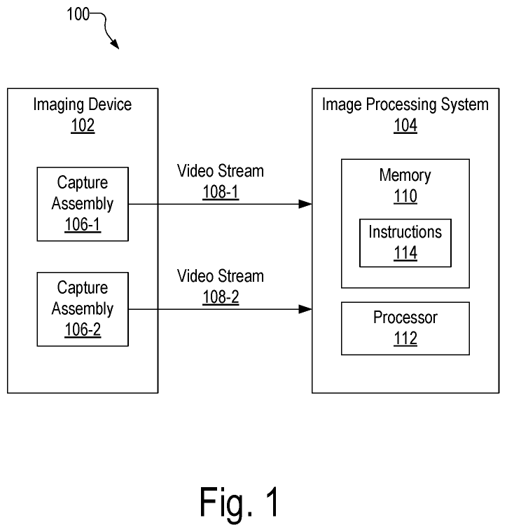

FIG. 1 shows an illustrative medical imaging system 100 configured to generate images of a scene during a medical procedure. In some examples, the scene may include a surgical area associated with a body on or within which the medical procedure is being performed (e.g., a body of a live animal, a human or animal cadaver, a portion of human or animal anatomy, tissue removed from human or animal anatomies, non-tissue work pieces, training models, etc.).

As shown, medical imaging system 100 includes an imaging device 102 in communication with an image processing system 104. Medical imaging system 100 may include additional or alternative components as may serve a particular implementation. In some examples, medical imaging system 100 or certain components of medical imaging system 100 may be implemented by a computer-assisted medical system.

Imaging device 102 may be implemented by an endoscope or other suitable device configured to generate video streams. For example, as shown, imaging device 102 may include a first capture assembly 106-1 and a second capture assembly 106-2 (collectively, “capture assemblies 106” or “image capture assemblies 106”) configured to output a first video stream 108-1 and a second video stream 108-2, respectively. Video streams 108-1 and 108-2 are referred to herein collectively as “video streams 108”. Each capture assembly 106 may include a lens, processing circuitry, and/or other components configured to capture and output a video stream. In some examples, capture assemblies 106 are configured to generate video streams 108 concurrently. In some examples, video streams 108 are combinable to create a stereoscopic viewing experience for a user. For example, video stream 108-1 may be a left stereo capture stream and video stream 108-2 may be a right stereo capture stream combinable to create a stereoscopic viewing experience for a user.

As used herein, a video stream may include a sequence of image frames (also referred to herein as images) of a scene captured by a capture assembly 106 of imaging device 102. The image frames may include one or more visible light image frames (i.e., one or more images acquired using visible light illumination) and/or one or more alternate imaging modality frames (e.g., one or more images acquired using non-visible light). Illustrative alternate imaging modality frames include fluorescence images acquired using fluorescence excitation illumination having wavelengths in a near-infrared light region.

Image processing system 104 may be configured to access (e.g., receive) video streams 108 and perform various operations with respect to the video stream, as described herein.

Image processing system 104 may be implemented by one or more computing devices and/or computer resources (e.g., processors, memory devices, storage devices, etc.) as may serve a particular implementation. As shown, image processing system 104 may include, without limitation, a memory 110 and a processor 112 selectively and communicatively coupled to one another. Memory 110 and processor 112 may each include or be implemented by computer hardware that is configured to store and/or process computer software. Various other components of computer hardware and/or software not explicitly shown in FIG. 1 may also be included within image processing system 104. In some examples, memory 110 and processor 112 may be distributed between multiple devices and/or multiple locations as may serve a particular implementation.

Memory 110 may store and/or otherwise maintain executable data used by processor 112 to perform any of the functionality described herein. For example, memory 110 may store instructions 114 that may be executed by processor 112. Memory 110 may be implemented by one or more memory or storage devices, including any memory or storage devices described herein, that are configured to store data in a transitory or non-transitory manner. Instructions 114 may be executed by processor 112 to cause image processing system 104 to perform any of the functionality described herein. Instructions 114 may be implemented by any suitable application, software, code, and/or other executable data instance. Additionally, memory 110 may also maintain any other data accessed, managed, used, and/or transmitted by processor 112 in a particular implementation.

Processor 112 may be implemented by one or more computer processing devices, including general purpose processors (e.g., central processing units (CPUs), graphics processing units (GPUs), microprocessors, etc.), special purpose processors (e.g., application specific integrated circuits (ASICs), field-programmable gate arrays (FPGAs), etc.), image signal processors, or the like. Using processor 112 (e.g., when processor 112 is directed to perform operations represented by instructions 114 stored in memory 110), image processing system 104 may perform various operations as described herein.

Various implementations of image processing system 104 will now be described with reference to the figures. The various modules illustrated in these figures as being included in image processing system 104 may be implemented by any suitable combination of hardware and/or software. As such, the modules represent various functions that may be performed by image processing system 104 alone or in combination with any of the other functions described herein as being performed by image processing system 104 and/or a component thereof.

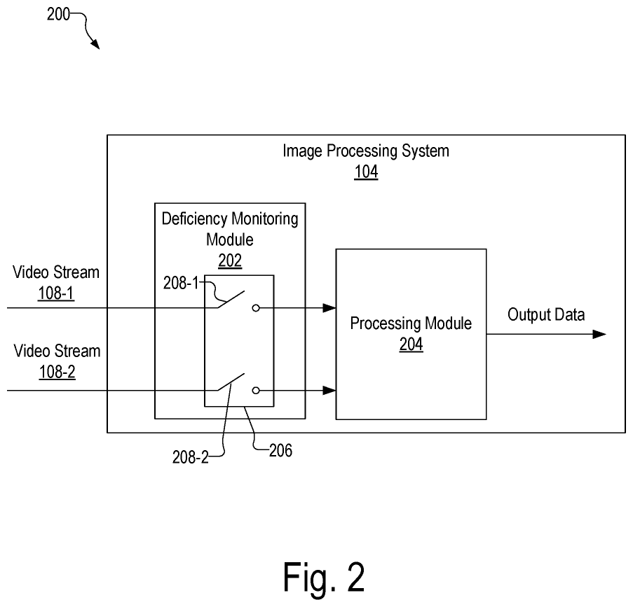

FIG. 2 shows an illustrative implementation 200 of image processing system 104. As shown, image processing system 104 may be configured to apply video streams 108 to a deficiency monitoring module 202 (“monitoring module 202”). Monitoring module 202 may be configured to monitor video streams 108 for one or more deficiencies. Examples of this are described herein.

Based on the monitoring, monitoring module 202 may selectively apply one or both of video streams 108 as an input to a processing module 204. This selective application of one or both video streams 108 as an input to a processing module 204 is illustrated in FIG. 2 as a selection block 206 including first and second switches 208-1 and 208-2 (collectively “switches 208”). As described herein, to selectively apply video stream 108-1 to processing module 204, monitoring module 202 may close switch 208-1. Likewise, to selectively apply video stream 108-2 to processing module 204, monitoring module 202 may close switch 208-2. While switches 208 are shown in the figures and used in the examples described herein, it will be recognized that switches 208 are merely illustrative of the many different ways in which monitoring module 202 may selectively apply one or both of video streams 108 to processing module 204. For example, any suitable hardware and/or software implementation of a state machine may be used to selectively apply one or both of video streams 108 to processing module 204.

As shown, processing module 204 may be configured to generate, based on the input, output data. The output data may be used to perform an image-based operation associated with the medical procedure. Illustrative image-based operations that may be performed by processing module 204 are described herein.

In some examples, monitoring module 202 may selectively apply one or both of video streams 108 to processing module 204 based on detecting a deficiency associated with one or both of video streams 108. Monitoring module 202 may detect a deficiency associated with a video stream (e.g., either of video streams 108) in any suitable manner.

For example, monitoring module 202 may detect a deficiency associated with a video stream based on an analysis of the video stream itself. For example, monitoring module 202 may detect a deficiency associated with a video stream by determining that an image frame within the video stream depicts an occlusion that blocks a field of view of a lens of imaging device 102. This determination may be made using any suitable image analysis algorithm (e.g., a machine learning model). The occlusion may be of any suitable type. For example, the occlusion may be caused by debris on the lens and/or smoke within a field of view of the lens.

As another example, an analysis of a video stream by monitoring module 202 may result in monitoring module 202 identifying an anomaly in the video stream. The anomaly may be caused by a rendering error, an error in image device operation, saturation within one or more image frames, etc. Based on this, monitoring module 202 may determine that a deficiency is associated with the video stream.

To illustrate, monitoring module 202 may determine that a threshold percentage of an image frame within a video stream depicts an occlusion that blocks a field of view of a lens of the imaging device. The threshold percentage may be set to any suitable value. Based on this determination, monitoring module 202 may determine that the video stream has a deficiency.

As another example, monitoring module 202 may determine that a particular pixel area of an image frame within the first video stream depicts an occlusion that blocks a field of view of a lens of the imaging device. The particular pixel area may be, for example, in a middle portion of the image frame. Based on this determination, monitoring module 202 may determine that the video stream has a deficiency.

Additionally or alternatively, monitoring module 202 may detect a deficiency associated with a video stream based on an analysis of the output data output by processing module 204. For example, monitoring module 202 may detect a deficiency associated with the output data by, for example, determining that a confidence interval associated with the output data is below a threshold. Based on this determination, monitoring module 202 may determine that a likely cause of the deficiency associated with the output data is caused by a deficiency associated with a video stream being applied as an input to processing module 204.

The image-based operation performed based on the output data output by processing module 202 may be performed by processing module 202 and/or one or more other computing components and may include any suitable operation as may serve a particular implementation. For example, the image-based operation may include determining (e.g., by using a machine learning model) a content classification of an image frame included in a video stream. For example, if the medical procedure is performed with respect to a patient, the content classification may indicate whether the image frame is an ex-body frame that depicts content external to a body of the patient or an in-body frame that does not depict content external to the body of the patient. Based on the content classification, an operation may be performed (e.g., by processing module 202) with respect to the image frame. Additionally or alternatively, the image-based operation may include an object identification operation and/or any other operation that uses images captured by an imaging device.

Various video stream selection heuristics that may be performed by image processing system 104 (e.g., monitoring module 202) will now be described. In these examples, various methods are illustrated by flowcharts. Each of these methods may be performed by image processing system 104 and/or any implementation thereof. Moreover, while the flowcharts depict illustrative operations according to one embodiment, other embodiments may omit, add to, reorder, and/or modify any of the operations shown in the flowcharts. Each of the operations shown in the flowcharts may be performed in any of the ways described herein.

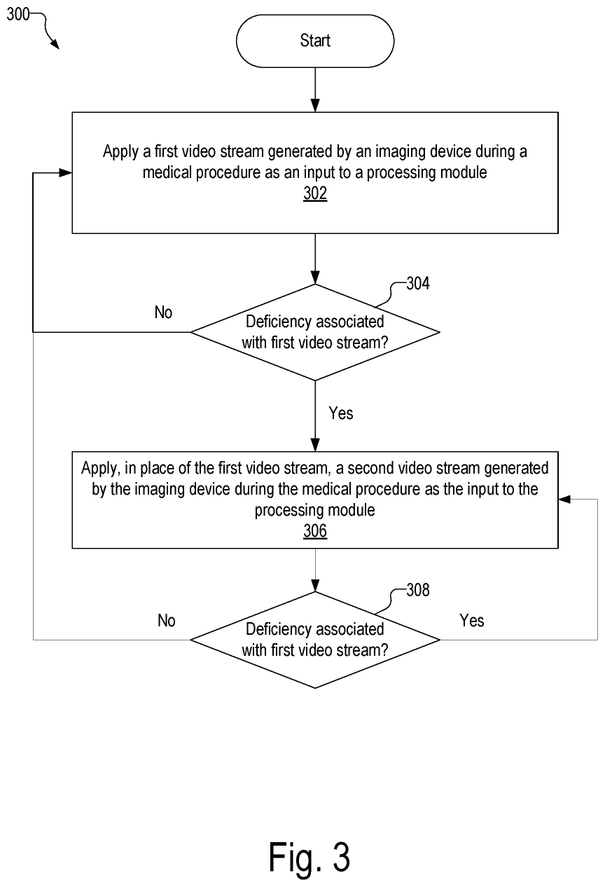

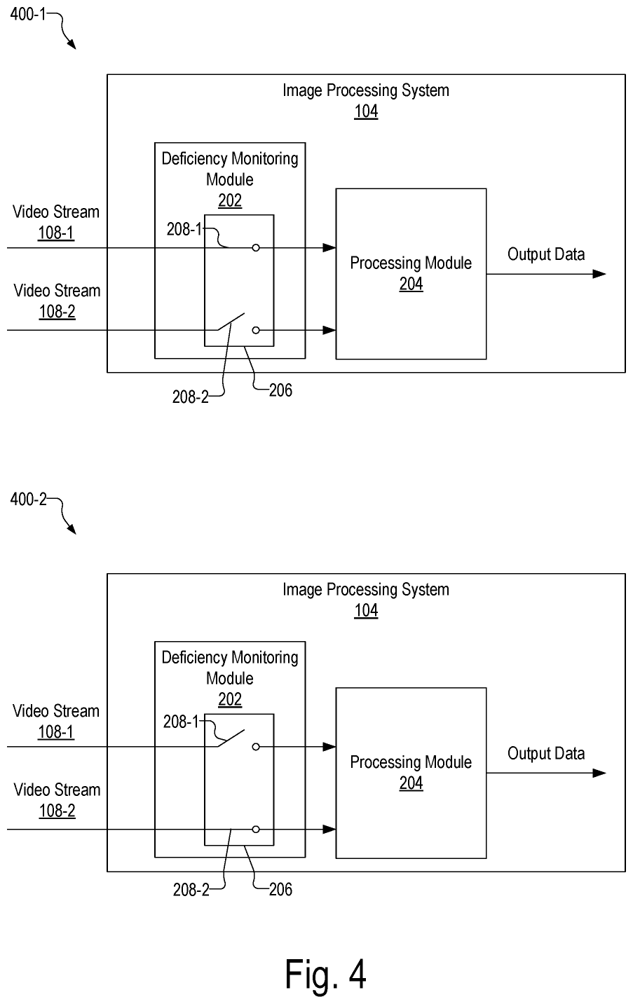

FIGS. 3-4 show an illustrative video stream selection heuristic that may be used when processing module 204 implements a mono algorithm (e.g., a processing algorithm that only needs one video stream as an input). In particular, FIG. 3 shows an illustrative method 300 that may be performed by image processing system 104 when processing module 204 implements a mono algorithm and FIG. 4 shows exemplary configurations 400-1 and 400-2 of image processing system 104 that illustrate various video stream selection states described in method 300.

With reference to FIG. 3, at operation 302, image processing system 104 may apply a first video stream generated by an imaging device during a medical procedure as an input to a processing module. This video stream selection state is illustrated in configuration 400-1. As shown, switch 208-1 is closed, thereby allowing video stream 108-1 to be provided as an input to processing module 204. In contrast, switch 208-2 is open, thereby preventing video stream 108-2 from being provided as an input to processing module 204. In this state, processing module 204 may perform the mono algorithm using only video stream 108-1.

While the first video stream is being applied as an input to the processing module, image processing system 104 may monitor the first video stream to determine if a deficiency is associated with the first video stream (decision 304).

If a deficiency is not detected within the first video stream (No, decision 304), image processing system 104 may continue applying the first video stream to the processing module (operation 302).

However, if a deficiency is detected within the first video stream (Yes, decision 304), image processing system 104 may apply, in place of the first video stream, a second video stream generated by the imaging device during the medical procedure as the input to the processing module (operation 306). In other words, image processing system 104 may abstain from applying the first video stream to the processing module and instead apply the second video stream to the processing module.

This video stream selection state is illustrated in configuration 400-2. As shown in configuration 400-2, switch 208-1 is open, thereby preventing video stream 108-1 from being provided as an input to processing module 204. In contrast, switch 208-2 is closed, thereby allowing video stream 108-2 to be provided as an input to processing module 204. In this state, processing module 204 may perform the mono algorithm using only video stream 108-2.

While the second video stream is being applied as an input to the processing module, image processing system 104 may continue to monitor the first video stream to determine if the deficiency is still associated with the first video stream (decision 308). If the deficiency is still associated with the first video stream (Yes, decision 308), image processing system 104 may continue applying the second video stream to the processing module (operation 306). However, if the deficiency is no longer associated with the first video stream (e.g., a lens used the capture the first video stream is no longer occluded) (No, decision 304), image processing system 104 may resume applying the first video stream to the processing module (operation 302) and stop applying the second video stream to the processing module.

In some examples, while the second video stream is being applied as an input to the processing module, image processing system 104 may determine that a deficiency is also associated with the second video stream (such that both video streams have deficiencies). In this example, image processing system 104 may abstain from providing either video stream to the processing module and/or otherwise disable the processing module.

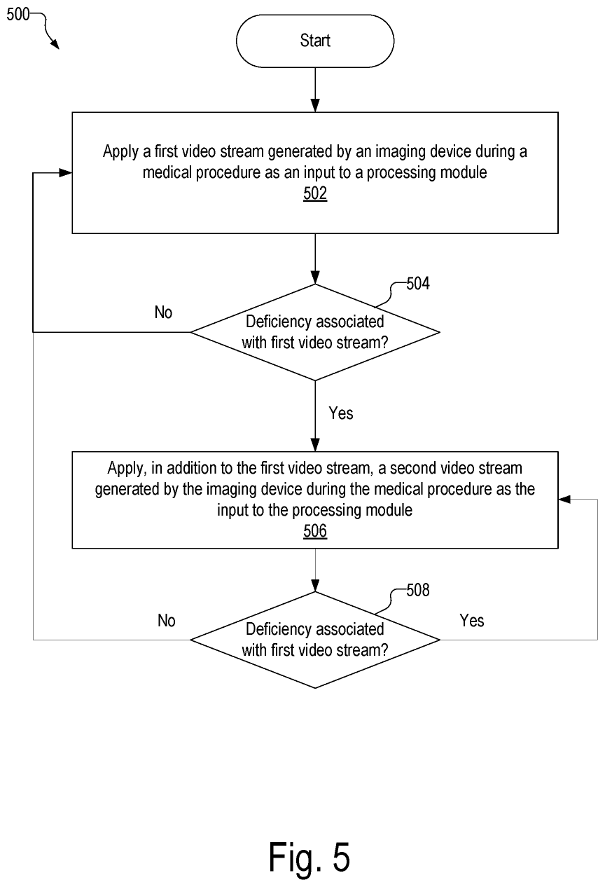

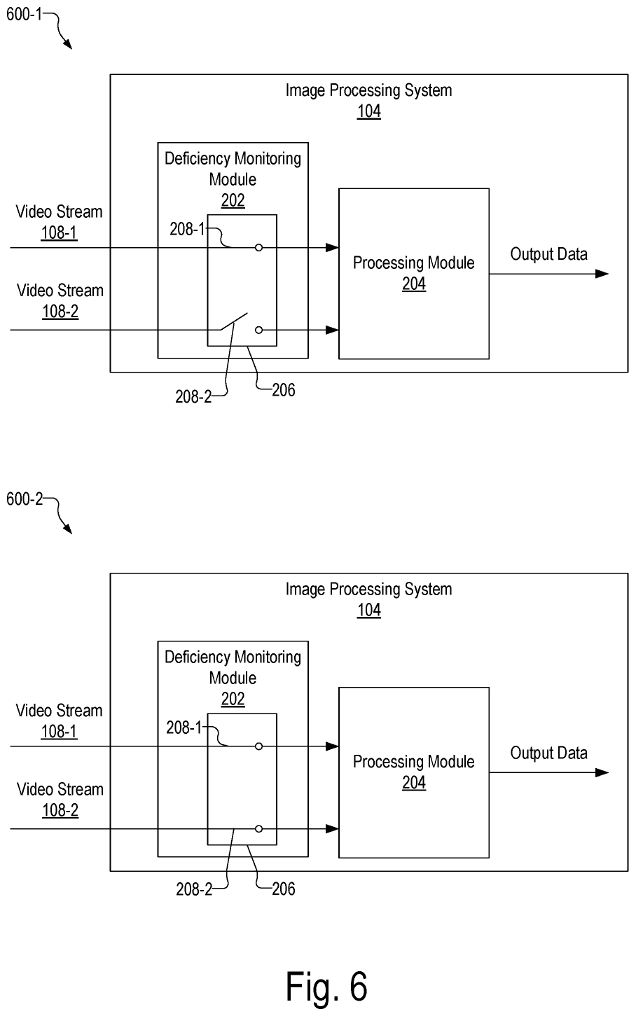

FIGS. 5-6 show another illustrative video stream selection heuristic that may be used when processing module 204 implements a mono algorithm. In particular, FIG. 5 shows an illustrative method 500 that may be performed by image processing system 104 when processing module 204 implements a mono algorithm and FIG. 6 shows exemplary configurations 600-1 and 600-2 of image processing system 104 that illustrate various video stream selection states described in method 500.

With reference to FIG. 5, at operation 502, image processing system 104 may apply a first video stream generated by an imaging device during a medical procedure as an input to a processing module. This video stream selection state is illustrated in configuration 600-1, which is similar to configuration 400-1 shown in FIG. 4. In this state, processing module 204 may perform the mono algorithm using only video stream 108-1.

While the first video stream is being applied as an input to the processing module, image processing system 104 may monitor the first video stream to determine if a deficiency is associated with the first video stream (decision 504).

If a deficiency is not detected within the first video stream (No, decision 504), image processing system 104 may continue applying the first video stream to the processing module (operation 502).

However, if a deficiency is detected within the first video stream (Yes, decision 504), image processing system 104 may apply, in addition to the first video stream, a second video stream generated by the imaging device during the medical procedure as the input to the processing module (operation 506). In other words, image processing system 104 may provide both the first and second video streams to the processing module.

This video stream selection state is illustrated in configuration 600-2. As shown in configuration 600-2, both switches 208-1 and 208-2 are closed thereby allowing both video streams 108-1 and 108-2 to be provided as an input to processing module 204. In this state, processing module 204 may perform the mono algorithm using both video streams 108, even though the mono algorithm only needs one of the video streams. For example, processing module 204 may use video stream 108-2 to validate output data generated using video stream 108-1, selectively use output data based on whichever stream produces more optimal results, etc.

While the second video stream is being applied as an input to the processing module, image processing system 104 may continue to monitor the first video stream to determine if the deficiency is still associated with the first video stream (decision 508). If the deficiency is still associated with the first video stream (Yes, decision 508), image processing system 104 may continue applying the second video stream to the processing module (operation 502). However, if the deficiency is no longer associated with the first video stream (e.g., a lens used the capture the first video stream is no longer occluded) (No, decision 504), image processing system 104 may resume applying the first video stream to the processing module in place of the second video stream (operation 502).

While both video streams are being applied as an input to processing module 204, image processing system 104 may continue to monitor the first video stream to determine if the deficiency is still associated with the first video stream (decision 508). If the deficiency is still associated with the first video stream (Yes, decision 508), image processing system 104 may continue applying the second video stream to the processing module (operation 502). However, if the deficiency is no longer associated with the first video stream (e.g., a lens used the capture the first video stream is no longer occluded) (No, decision 504), image processing system 104 may resume applying the first video stream to the processing module (operation 502) and stop applying the second video stream to the processing module.

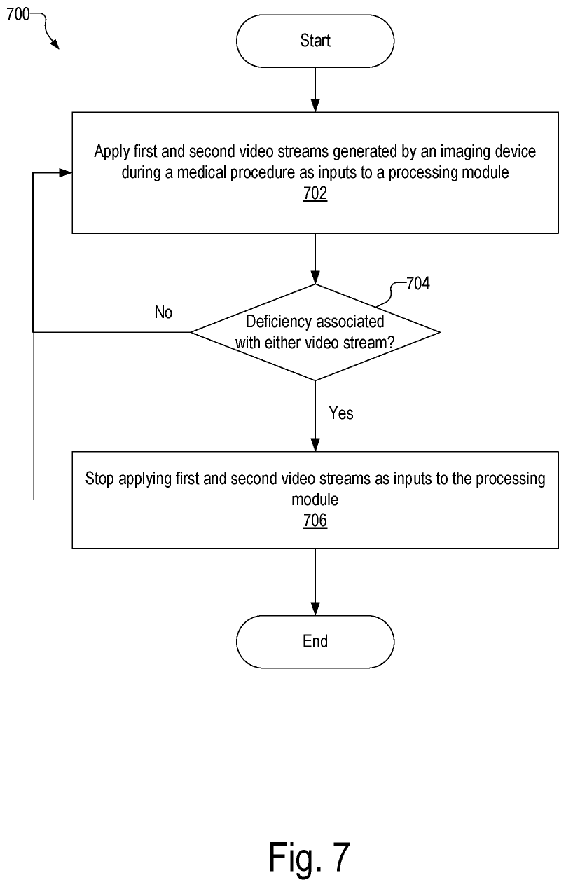

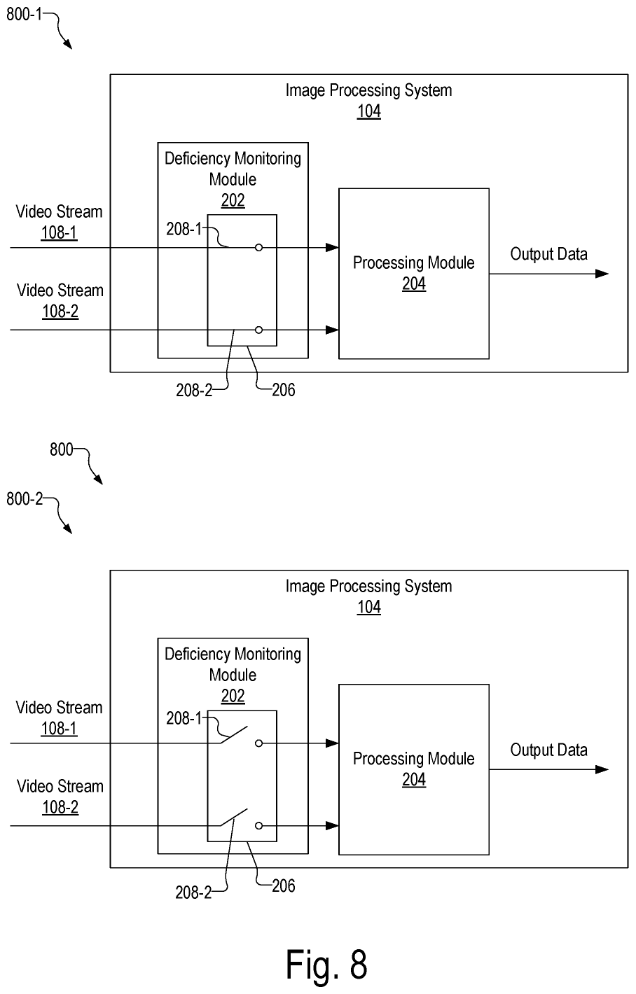

FIGS. 7-8 show an illustrative video stream selection heuristic that may be used when processing module 204 implements a stereo algorithm (e.g., an algorithm that needs both video streams). In particular, FIG. 7 shows an illustrative method 700 that may be performed by image processing system 104 when processing module 204 implements a stereo algorithm and FIG. 8 shows exemplary configurations 800-1 and 800-2 of image processing system 104 that illustrate various video stream selection states described in method 700.

With reference to FIG. 7, at operation 702, image processing system 104 may apply first and second video streams generated by an imaging device during a medical procedure as inputs to a processing module. This video stream selection state is illustrated in configuration 800-1, which shows both switches 208 in a closed position, thereby allowing both video streams 108 to be applied as inputs to processing module 204.

While the video streams are being applied as inputs to the processing module, image processing system 104 may monitor both video streams to determine if a deficiency is associated with either of the video streams (decision 704).

If a deficiency is not detected within either video stream (No, decision 704), image processing system 104 may continue applying the video streams to the processing module (operation 702).

However, if a deficiency is detected with respect to either video stream (Yes, decision 704), image processing system 104 may stop applying the first and second video streams as inputs to the processing module (operation 706). This video stream selection state is illustrated in configuration 800-2, which shows both switches 208 in an open position, thereby preventing both video streams 108 from being applied as inputs to processing module 204.

In some alternative embodiments, image processing system 104 may direct processing module 204 to perform a mono algorithm with one of the video streams in response to detecting a deficiency with respect to the other video stream.

In response to detecting a deficiency associated with one or both video streams 108, image processing system 104 may perform one or more remedial actions with respect to the deficiency. For example, image processing system 104 may provide a notification regarding the deficiency and/or perform an operation configured to remedy the deficiency.

For example, image processing system 104 may determine a type of an occlusion that cases a deficiency within a video stream. This determination may be made using any suitable image analysis and/or classification algorithm (e.g., a machine learning model). Based on the determined type, image processing system 104 may perform a remedial action configured to remedy the deficiency.

To illustrate, image processing system 104 may determine that the occlusion includes or is caused by debris on a lens of imaging device 102. Based on this determination, image processing system 104 may initiate a cleaning operation with respect to the lens. The cleaning operation may be performed in any suitable manner.

As another example, image processing system 104 may determine that the occlusion includes or is caused by smoke (e.g., smoke caused by a cautery tool) within the field of view of the lens. Based on this determination, image processing system 104 may initiate a smoke evacuation operation. The smoke evacuation operation may be performed in any suitable manner.

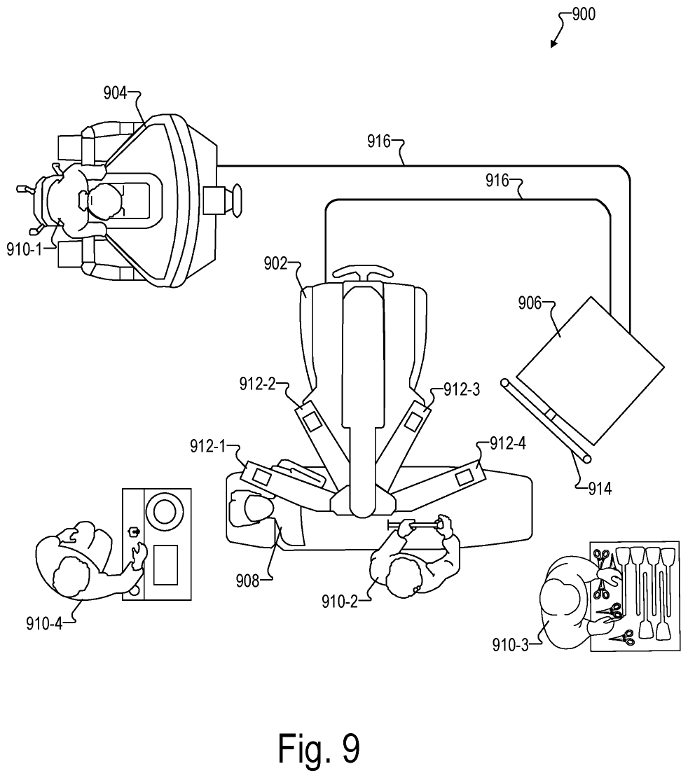

As has been described, imaging device 102 and/or image processing system 104 may be associated in certain examples with a computer-assisted medical system used to perform a medical procedure on a body. To illustrate, FIG. 9 shows an illustrative computer-assisted medical system 900 that may be used to perform various types of medical procedures including surgical and/or non-surgical procedures.

As shown, computer-assisted medical system 900 may include a manipulator assembly 902 (a manipulator cart is shown in FIG. 9), a user control apparatus 904, and an auxiliary apparatus 906, all of which are communicatively coupled to each other. Computer-assisted medical system 900 may be utilized by a medical team to perform a computer-assisted medical procedure or other similar operation on a body of a patient 908 or on any other body as may serve a particular implementation. As shown, the medical team may include a first user 910-1 (such as a surgeon for a surgical procedure), a second user 910-2 (such as a patient-side assistant), a third user 910-3 (such as another assistant, a nurse, a trainee, etc.), and a fourth user 910-4 (such as an anesthesiologist for a surgical procedure), all of whom may be collectively referred to as users 910, and each of whom may control, interact with, or otherwise be a user of computer-assisted medical system 900. More, fewer, or alternative users may be present during a medical procedure as may serve a particular implementation. For example, team composition for different medical procedures, or for non-medical procedures, may differ and include users with different roles.

While FIG. 9 illustrates an ongoing minimally invasive medical procedure such as a minimally invasive surgical procedure, it will be understood that computer-assisted medical system 900 may similarly be used to perform open medical procedures or other types of operations. For example, operations such as exploratory imaging operations, mock medical procedures used for training purposes, and/or other operations may also be performed.

As shown in FIG. 9, manipulator assembly 902 may include one or more manipulator arms 912 (e.g., manipulator arms 912-1 through 912-4) to which one or more instruments may be coupled. The instruments may be used for a computer-assisted medical procedure on patient 908 (e.g., in a surgical example, by being at least partially inserted into patient 908 and manipulated within patient 908). While manipulator assembly 902 is depicted and described herein as including four manipulator arms 912, it will be recognized that manipulator assembly 902 may include a single manipulator arm 912 or any other number of manipulator arms as may serve a particular implementation. While the example of FIG. 9 illustrates manipulator arms 912 as being robotic manipulator arms, it will be understood that, in some examples, one or more instruments may be partially or entirely manually controlled, such as by being handheld and controlled manually by a person. For instance, these partially or entirely manually controlled instruments may be used in conjunction with, or as an alternative to, computer-assisted instrumentation that is coupled to manipulator arms 912 shown in FIG. 9.

During the medical operation, user control apparatus 904 may be configured to facilitate teleoperational control by user 910-1 of manipulator arms 912 and instruments attached to manipulator arms 912. To this end, user control apparatus 904 may provide user 910-1 with imagery of an operational area associated with patient 908 as captured by an imaging device. To facilitate control of instruments, user control apparatus 904 may include a set of master controls. These master controls may be manipulated by user 910-1 to control movement of the manipulator arms 912 or any instruments coupled to manipulator arms 912.

Auxiliary apparatus 906 may include one or more computing devices configured to perform auxiliary functions in support of the medical procedure, such as providing insufflation, electrocautery energy, illumination or other energy for imaging devices, image processing, or coordinating components of computer-assisted medical system 900. In some examples, auxiliary apparatus 906 may be configured with a display monitor 914 configured to display one or more user interfaces, or graphical or textual information in support of the medical procedure. In some instances, display monitor 914 may be implemented by a touchscreen display and provide user input functionality. Augmented content provided by a region-based augmentation system may be similar, or differ from, content associated with display monitor 914 or one or more display devices in the operation area (not shown).

Manipulator assembly 902, user control apparatus 904, and auxiliary apparatus 906 may be communicatively coupled one to another in any suitable manner. For example, as shown in FIG. 9, manipulator assembly 902, user control apparatus 904, and auxiliary apparatus 906 may be communicatively coupled by way of control lines 916, which may represent any wired or wireless communication link as may serve a particular implementation. To this end, manipulator assembly 902, user control apparatus 904, and auxiliary apparatus 906 may each include one or more wired or wireless communication interfaces, such as one or more local area network interfaces, Wi-Fi network interfaces, cellular interfaces, and so forth.

In certain embodiments, one or more of the processes described herein may be implemented at least in part as instructions embodied in a non-transitory computer-readable medium and executable by one or more computing devices. In general, a processor (e.g., a microprocessor) receives instructions, from a non-transitory computer-readable medium, (e.g., a memory, etc.), and executes those instructions, thereby performing one or more processes, including one or more of the processes described herein. Such instructions may be stored and/or transmitted using any of a variety of known computer-readable media.

A computer-readable medium (also referred to as a processor-readable medium) includes any non-transitory medium that participates in providing data (e.g., instructions) that may be read by a computer (e.g., by a processor of a computer). Such a medium may take many forms, including, but not limited to, non-volatile media, and/or volatile media. Non-volatile media may include, for example, optical or magnetic disks and other persistent memory. Volatile media may include, for example, dynamic random access memory (“DRAM”), which typically constitutes a main memory. Common forms of computer-readable media include, for example, a disk, hard disk, magnetic tape, any other magnetic medium, a compact disc read-only memory (“CD-ROM”), a digital video disc (“DVD”), any other optical medium, random access memory (“RAM”), programmable read-only memory (“PROM”), electrically erasable programmable read-only memory (“EPROM”), FLASH-EEPROM, any other memory chip or cartridge, or any other tangible medium from which a computer can read.

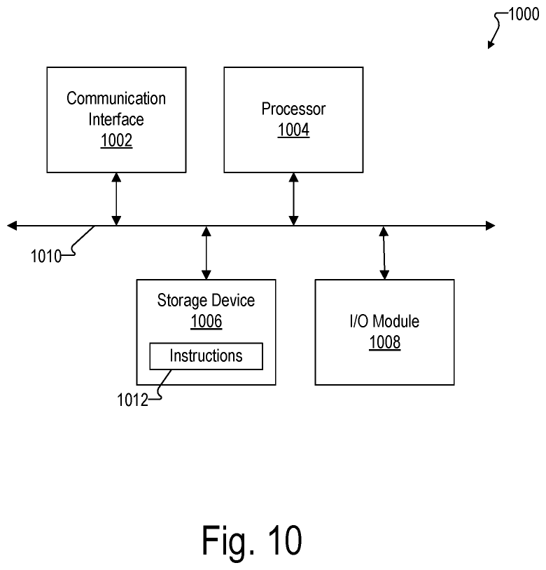

FIG. 10 shows an illustrative computing device 1000 that may be specifically configured to perform one or more of the processes described herein. Any of the systems, computing devices, and/or other components described herein may be implemented by computing device 1000.

As shown in FIG. 10, computing device 1000 may include a communication interface 1002, a processor 1004, a storage device 1006, and an input/output (“I/O”) module 1008 communicatively connected one to another via a communication infrastructure 1010. While an illustrative computing device 1000 is shown in FIG. 10, the components illustrated in FIG. 10 are not intended to be limiting. Additional or alternative components may be used in other embodiments. Components of computing device 1000 shown in FIG. 10 will now be described in additional detail.

Communication interface 1002 may be configured to communicate with one or more computing devices. Examples of communication interface 1002 include, without limitation, a wired network interface (such as a network interface card), a wireless network interface (such as a wireless network interface card), a modem, an audio/video connection, and any other suitable interface.

Processor 1004 generally represents any type or form of processing unit capable of processing data and/or interpreting, executing, and/or directing execution of one or more of the instructions, processes, and/or operations described herein. Processor 1004 may perform operations by executing computer-executable instructions 1012 (e.g., an application, software, code, and/or other executable data instance) stored in storage device 1006.

Storage device 1006 may include one or more data storage media, devices, or configurations and may employ any type, form, and combination of data storage media and/or device. For example, storage device 1006 may include, but is not limited to, any combination of the non-volatile media and/or volatile media described herein. Electronic data, including data described herein, may be temporarily and/or permanently stored in storage device 1006. For example, data representative of computer-executable instructions 1012 configured to direct processor 1004 to perform any of the operations described herein may be stored within storage device 1006. In some examples, data may be arranged in one or more databases residing within storage device 1006.

I/O module 1008 may include one or more I/O modules configured to receive user input and provide user output. I/O module 1008 may include any hardware, firmware, software, or combination thereof supportive of input and output capabilities. For example, I/O module 1008 may include hardware and/or software for capturing user input, including, but not limited to, a keyboard or keypad, a touchscreen component (e.g., touchscreen display), a receiver (e.g., an RF or infrared receiver), motion sensors, and/or one or more input buttons.

I/O module 1008 may include one or more devices for presenting output to a user, including, but not limited to, a graphics engine, a display (e.g., a display screen), one or more output drivers (e.g., display drivers), one or more audio speakers, and one or more audio drivers. In certain embodiments, I/O module 1008 is configured to provide graphical data to a display for presentation to a user. The graphical data may be representative of one or more graphical user interfaces and/or any other graphical content as may serve a particular implementation.

In the preceding description, various exemplary embodiments have been described with reference to the accompanying drawings. It will, however, be evident that various modifications and changes may be made thereto, and additional embodiments may be implemented, without departing from the scope of the invention as set forth in the claims that follow. For example, certain features of one embodiment described herein may be combined with or substituted for features of another embodiment described herein. The description and drawings are accordingly to be regarded in an illustrative rather than a restrictive sense.

1. A system comprising:
a memory storing instructions; and
a processor communicatively coupled to the memory and configured to execute the instructions to:
apply, as an input to a processing module, a first video stream generated by an imaging device during a medical procedure, the processing module configured to generate, based on the input, output data used to perform an image-based operation associated with the medical procedure;
detect, while the first video stream is being applied to the processing module, a deficiency associated with the first video stream; and
apply, as the input to the processing module and based on the detecting of the deficiency, a second video stream generated by the imaging device during the medical procedure.

2. The system of claim 1, wherein the processor is further configured to execute the instructions to abstain from applying the first video stream as the input to the processing module while the second video stream is being applied as the input to the processing module.

3. The system of claim 1, wherein the processor is further configured to execute the instructions to continue applying the first video stream as the input to the processing module while the second video stream is being applied as the input to the processing module.

4. The system of claim 1, wherein the detecting the deficiency associated with the first video stream comprises determining that an image frame within the first video stream depicts an occlusion that blocks a field of view of a lens of the imaging device.

5. The system of claim 4, wherein the processor is further configured to execute the instructions to:
determine a type of the occlusion; and
perform, based on the determined type, a remedial action with respect to the occlusion.

6. The system of claim 5, wherein:
the determining the type of the occlusion comprises determining that the occlusion comprises debris on the lens; and
the performing the remedial action with respect to the occlusion comprises initiating a cleaning operation with respect to the lens.

7. The system of claim 5, wherein:
the determining the type of the occlusion comprises determining that the occlusion comprises smoke within the field of view of the lens; and
the performing the remedial action with respect to the occlusion comprises initiating a smoke evacuation operation.

8. The system of claim 1, wherein the processor is further configured to execute the instructions to perform a remedial action with respect to the deficiency associated with the first video stream.

9. The system of claim 8, wherein the remedial action comprises one or more of providing a notification regarding the deficiency, initiating a smoke evacuation operation, or initiating a lens cleaning operation.

10. The system of claim 1, wherein the detecting the deficiency associated with the first video stream comprises determining that a threshold percentage of an image frame within the first video stream depicts an occlusion that blocks a field of view of a lens of the imaging device.

11. The system of claim 1, wherein the detecting the deficiency associated with the first video stream comprises determining that a particular pixel area of an image frame within the first video stream depicts an occlusion that blocks a field of view of a lens of the imaging device.

12. The system of claim 1, wherein the processor is further configured to execute the instructions to:
detect, while the second video stream is being applied to the processing module, an additional deficiency associated with the second video stream; and
abstain, based on the detecting of the deficiency associated with the first video stream and the additional deficiency associated with the second video stream, from applying the first and second video streams to the processing module.

13. The system of claim 1, wherein the processor is further configured to execute the instructions to:
determine, while the second video stream is being applied to the processing module, that the deficiency is no longer associated with the first video stream; and
stop, based on the determining that the deficiency is no longer associated with the first video stream, the applying of the second video stream as the input to the processing module.

14. The system of claim 1, wherein:
the processor is further configured to execute the instructions to detect a deficiency associated with the output data; and
the detecting the deficiency associated with the first video stream is based on the detecting the deficiency associated with the output data.

15. The system of claim 14, wherein the detecting of the deficiency associated with the output data comprises determining that a confidence interval associated with the output data is below a threshold.

16. (canceled)

17. The system of claim 1, wherein the performing of the image-based operation comprises determining a content classification of an image frame included in the first video stream.

18. The system of claim 17, wherein:
the medical procedure is performed with respect to a patient; and
the content classification indicates whether the image frame is an ex-body frame that depicts content external to a body of the patient or an in-body frame that does not depict content external to the body of the patient.

19-22. (canceled)

23. A system comprising:
a memory storing instructions; and
a processor communicatively coupled to the memory and configured to execute the instructions to:
monitor first and second video streams generated by an imaging device during a medical procedure for one or more deficiencies, the first video stream generated by the imaging device using a first image capture assembly and the second video stream generated by the imaging device using a second image capture assembly; and
selectively use, based on the monitoring, one or more of the first video stream or the second video stream to perform an image-based operation associated with the medical procedure.

24. The system of claim 23, wherein the selectively using one or more of the first video stream or the second video stream to perform the image-based operation associated with the medical procedure comprises:
selectively applying one or more of the first video stream or the second video stream as an input to a processing module, the processing module configured to generate output data based on the input; and
perform, based on the output data, an operation associated with the medical procedure.

25. A method comprising:
applying, by an image processing system as an input to a processing module, a first video stream generated by an imaging device during a medical procedure, the processing module configured to generate, based on the input, output data used to perform an image-based operation associated with the medical procedure;
detecting, by the image processing system while the first video stream is being applied to the processing module, a deficiency associated with the first video stream; and
applying, by the image processing system as the input to the processing module and based on the detecting of the deficiency, a second video stream generated by the imaging device during the medical procedure.

26-31. (canceled)
